(19) United States
(12) Patent Application Publication
(10) Pub. No.: US 2022/0406467 A1
(43) Pub. Date: Dec. 22, 2022

(54) ANONYMIZED HEALTH MONITORING PLATFORM

(71) Applicant: Oura Health Oy, Oulu (FI)

(72) Inventors: Brian Gilan, San Francisco, CA (US); Ville Saarinen, Helsinki (FI); Ossi Hanhinen, Espoo (FI); Oyelowo Oyedayo, Vantaa (FI); Janne Haapsaari, Vantaa (FI); Utkarsh Raut, Espoo (FI); Denis Vorotyntsev, Helsinki (FI); Kirstin Aschbacher, San Francisco, CA (US)

(21) Appl. No.: 17/807,010

(22) Filed: Jun. 15, 2022

Related U.S. Application Data
(60) Provisional application No. 63/211,506, filed on Jun. 16, 2021.

(51) Int. Cl. G16H 50/30; G16H 50/70; G06F 21/62

(52) U.S. Cl. CPC G16H 50/30; G16H 50/70; G06F 21/6254

(57) ABSTRACT

Methods, systems, and devices for health monitoring platform are described. The method may include receiving physiological data continuously collected from users via wearable devices, the users being associated with respective anonymized user identifiers. The method may include identifying health risk metrics associated with each respective user based on the received physiological data, and identifying a potential health risk for at least one anonymized user identifier corresponding to at least one user based on the health risk metrics associated with the at least one user satisfying a predefined thresholds. The method may include displaying a notification of the potential health risk associated with the at least one anonymized user identifier on an administrator user device, and displaying a configurable message associated with the potential health risk on an additional user device corresponding to the at least one user.

### CROSS REFERENCE

The present Application for Patent claims the benefit of U.S. Provisional Patent Application No. 63/211,506 by GILAN et al., entitled “ANONYMIZED HEALTH MONITORING PLATFORM,” filed Jun. 16, 2021, assigned to the assignee thereof, and expressly incorporated by reference herein.

### FIELD OF TECHNOLOGY

The following relates to wearable devices and data processing, including an anonymized health monitoring platform.

### BACKGROUND

Some wearable devices may be configured to collect physiological data from users, including temperature data, heart rate data, and the like. Many users have a desire for more insight regarding their physical health.

### BRIEF DESCRIPTION OF THE DRAWINGS

FIGS. 1 and 2 illustrate examples of systems that support an anonymized health monitoring platform in accordance with aspects of the present disclosure.

FIG. 3 illustrates an example of a health monitoring platform that supports an anonymized health monitoring platform in accordance with aspects of the present disclosure.

FIGS. 4-7 illustrate examples of graphical user interfaces (GUIs) that support anonymized health monitoring platform in accordance with aspects of the present disclosure.

FIG. 8 shows a block diagram of an apparatus that supports an anonymized health monitoring platform in accordance with aspects of the present disclosure.

FIG. 9 shows a block diagram of a wearable application that supports an anonymized health monitoring platform in accordance with aspects of the present disclosure.

FIG. 10 shows a diagram of a system including a device that supports an anonymized health monitoring platform in accordance with aspects of the present disclosure.

FIGS. 11 through 13 show flowcharts illustrating methods that support anonymized health monitoring platform in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, heart rate data, and the like. In order to efficiently and accurately track physiological data, a wearable device may be configured to collect data continuously while the user wears the device.

Most individuals do not have the capability to continually monitor their health. For example, most individuals go to the doctor a few times a year. As such, most individuals have several snapshots of their health-related data (e.g., temperature, heart rate, blood pressure, etc.) at a few points in time throughout the year. The limited data points, along with the infrequent and inconsistent times that the data points are collected, provide a limited view of the user's overall health. Moreover, these data points may often be taken after an individual knows they are sick, which further limits the utility of the collected data points. As such, techniques for health monitoring with infrequent snapshots of health-related data may be deficient for multiple reasons.

Accordingly, to facilitate improved health monitoring, aspects of the present disclosure are directed to a health monitoring platform configured to continuously collect physiological data from users to provide continuous health monitoring. Specifically, aspects of the present disclosure are directed to techniques for calculating health risk metrics (e.g., illness risk metrics) for users based on acquired data, and alerting users and/or administrators (e.g., doctors, coaches, managers, employers) of a potential health risk when a user exhibits a health risk metric that satisfies (e.g., exceeds) one or more predefined thresholds.

In some implementations, wearable devices may be used to continuously acquire physiological data and other data (e.g., sleep data) from a group of users for improved health monitoring. The respective users and/or an administrator of the group of users may have access to the acquired physiological data and/or scores and metrics that are determined based on the acquired physiological data (e.g., health risk metrics, Sleep Scores, Readiness Scores). In some implementations, users may be able to view their physiological data and/or calculated scores/metrics. Comparatively, in some cases, physiological data and/or calculated scores/metrics may be presented to administrators in an anonymized manner to improve the privacy of the respective users.

For example, an administrator associated with a group of users may access a user interface that displays the physiological data and/or scores/metrics (e.g., health risk metrics, Sleep Scores, Readiness Scores) for each user of the group. In some examples, the administrator may know the identity of each user. In some other examples, each of the physiological data and/or calculated metrics/scores for each user may be anonymized. For instance, physiological data and health risk scores for each user may be presented to the administrator along with an anonymized user identifier associated with each respective user. By anonymously presenting acquired physiological data and calculated scores/metrics, the privacy and security for each of the respective users may be preserved.

In some implementations, an administrator of a group of users may set or otherwise define thresholds for each metric or score, where the predefined thresholds may trigger messaging or other actions when the respective thresholds are satisfied. For example, if the health monitoring system determines that a health risk metric for a user satisfies (e.g., exceeds) a predefined threshold, thereby indicating a presence of a potential health risk, the system may automatically trigger transmission of a message to the user and/or an administrator. In cases with non-anonymized health monitoring, the health monitoring platform may tell the administrator which user is associated with the potential health risk so that the administrator may directly contact the user. Conversely, in cases with anonymized health monitoring, the administrator may not know the identity of the user associated with the potential health risk. However, in such cases, the administrator may see whether the user has viewed a message triggered by the identified health risk, and may decide to resend the message to the user after some time duration.

While much of the present disclosure is described in the context of health risk metrics related to illness, this is not to be regarded as a limitation of the present disclosure. Indeed, it is contemplated herein that aspects of the present disclosure may be used for continuously monitoring user health with respect to any potential health condition, including potential cardiac conditions/episodes, insomnia, atrial fibrillation, and the like. In particular, techniques described herein may enable the detection of illness prior to a user actually experiencing symptoms (e.g., pre-symptomatic illness detection), which may help reduce the spread of illness. Moreover, physiological data associated with a user may be used to update any score, measure, metric, or other abstraction associated with a user's health or activity. The health monitoring system may detect multiple health conditions, and may display information related to health metrics for each illness.

Aspects of the disclosure are initially described in the context of systems supporting physiological data collection from users via wearable devices. Additional aspects of the disclosure are described in the context of example graphical user interfaces (GUIs). Aspects of the disclosure are further illustrated by and described with reference to apparatus diagrams, system diagrams, and flowcharts that relate to an anonymized health monitoring platform.

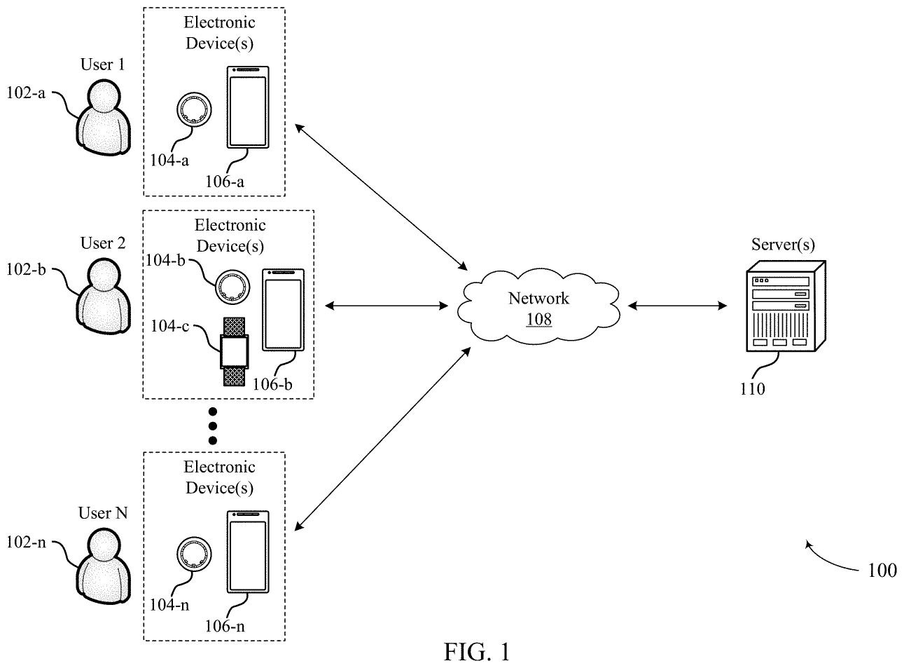

FIG. 1 illustrates an example of a system 100 that supports an anonymized health monitoring platform in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, which may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices, smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the respective devices of the system 100 may support techniques for monitoring health metrics related to illness by using a wearable device. In particular, the system 100 illustrated in FIG. 1 may support techniques for using a risk score for assessing illness likelihood of a user 102, and notifying the user 102 if the risk score surpasses an acceptable threshold. For example, as shown in FIG. 1, User 1 (e.g., user 102-a) may be associated with a wearable device (e.g., ring 104-a) and a user device 106-a. In this example, the ring 104-a may collect physiological data associated with the user, including temperature, heart rate, HRV, and the like. In some aspects, data collected by the ring 104-a may be used to determine whether User 1 is ill, or will become ill. For example, the data may be used to calculate a health risk metric indicating a relative probability that User 1 is ill, or will become ill (e.g., relative probability that User 1 will transition from a healthy state to an unhealthy state).

Detection of illness and calculation of a health risk metrics may be performed by any of the components of the system 100, including the ring 104-a, the user device 106-a associated with User 1, the one or more servers 110, or any combination thereof. The system 100 may be configured to perform pre-symptomatic illness detection, in which illness is identified or predicted prior to a user 102 experiencing symptoms (e.g., prior to symptom onset). The system 100 may detect one or more illnesses for the user 102. In some cases, calculated health risk metrics may be compared to predefined thresholds to perform the illness detection/health monitoring techniques described herein. In some cases, the predefined thresholds may be set or adjusted by an administrator based on a relative acceptable health risk metric for each user 102 in the system (e.g., User 1, User 2 . . . User N).

Upon detecting that a health risk metric for a user 102 satisfies a predefined threshold, the system 100 may notify the user 102 and/or a system administrator of one or more potential health risks. For example, upon detecting the health risk metric for a User 1 satisfies a predefined threshold, the system 100 may display a notification of the potential health risk via the user device 106-a. Moreover, the system 100 may monitor whether the User 1 viewed the notification of the potential health risk (e.g., via a GUI of the mobile device), and may selectively send a reminder accordingly (e.g., transmit a reminder or follow-up notification if the User 1 has not viewed the notification). In some implementations, the notifications of potential health risks associated with User 1 may be sent to User 1 (e.g., user device 106-a), an administrative personnel (e.g., manager of the group of users 102-a, 102-b, 102-c), or both. In some implementations, the administrator may know the identity of User 1, and may transmit the notification of the detected potential health risk to User 1 directly. In some other cases, health monitoring may be performed and reported to administrators anonymously such that the administrators may be able to view an anonymous list of users and corresponding health risk metrics. In such cases, the system 100 may notify User 1 and the administrator of the potential illness, where the identity of the user remains anonymous to the administrator. That is, administrator may view health risk metrics and/or physiological data for a group of users, but may not know what health risk metrics and/or physiological data corresponds to which user.

In some implementations, an administrator may view that one or more users 102 exhibit health risk metrics above a predefined threshold, and may view whether the users 102 have seen any notifications related to one or more potential health risks (e.g., one or more potential illnesses). In some implementations, the system 100 may generate the notification of potential health risks for User 1 (e.g., via the ring 104, user device 106, or both) based on the detected health risk metric, where the alerts indicate to the user 102 the potential health risks. The generated alerts may additionally or alternatively, provide other insights regarding the potential health risks, such as health trends for User 1, contributing factors for the detected health risks, whether User 1 should consider contacting a medical professional, whether the user should stay home from work or other activities, and the like.

Techniques described herein may support a health monitoring platform that enables anonymized health tracking for a group of users. In particular, techniques described herein may enable physiological data for a group of users to be continuously tracked in order to identify users who are likely to be sick, or likely to become sick in the near future. By notifying users and/or administrators (e.g., doctors, coaches, office managers, business owners) of potential illness, techniques described herein may reduce a spread of illness, and improve health tracking. Moreover, by providing notifications of health risk metrics in an anonymous manner, techniques described herein may enable administrators to track a risk of illness throughout a group of users while simultaneously maintaining a level of privacy with respect to physiological data acquired from the respective users.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

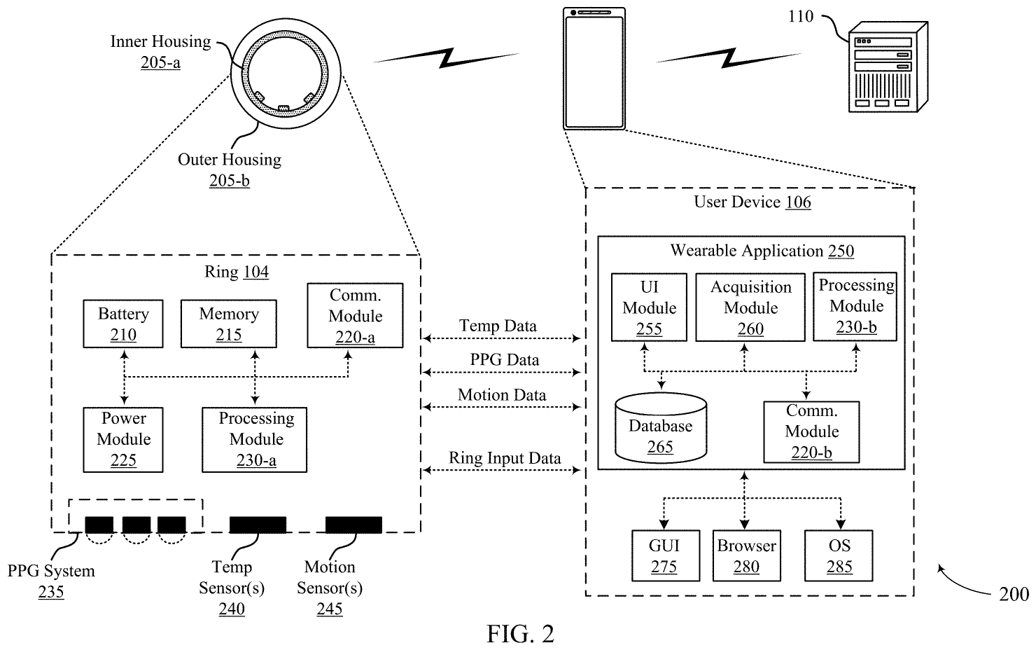

FIG. 2 illustrates an example of a system 200 that supports an anonymized health monitoring platform in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, which may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, which may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user's finger. In some implementations, the PPG system 235 may be configured as a transmissive PPG system 235 in which the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, which may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, rapid eye movement (REM) sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period. Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day.

The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the ring 104, user device 106, and servers 110 of the system 200 may be configured to evaluate physiological data and sleep patterns for a user 102. In particular, the respective components of the system 200 may be used to determine/predict potential illness for the user based on the acquired physiological data and sleep patterns for the user. In particular, the system 200 may be configured to detect/predict illness onset within users 102 prior to the respective users 102 experiencing any noticeable symptoms of illness. This pre-symptomatic illness detection may reduce the severity of illness, and reduce the spread of illness within a population.

For example, as noted previously herein, the ring 104 of the system 200 may be worn by a user to collect physiological data from the user, including temperature data, heart rate data, HRV data, and the like. Data collected by the ring 104 may be used to determine scores (e.g., Sleep Scores, Readiness Scores) for the user for each sleep day. Additionally or alternatively, data collected by the ring 104 may be used to determine one or more health risk metrics for the user related to potential illness and/or other health concerns. For example, the system 200 may determine an illness risk metric (e.g., risk score) for a given day (e.g., which may be a 24 hour period of continuous monitoring), where the illness risk metric is associated with a relative probability that the user will become ill with any number of illnesses or other health concerns (e.g., transition from a healthy state to an unhealthy state). In some aspects, health risk metrics may be calculated for the user for each respective day, such that a first day is associated with a first set of health risk metrics, and a second day is associated with a second set of health risk metrics. Health risk metrics may be calculated for each day based on data collected by the ring 104 during the respective day. Health risk metrics may include, but are not limited to, risk scores, health scores, illness risk metrics, and the like.

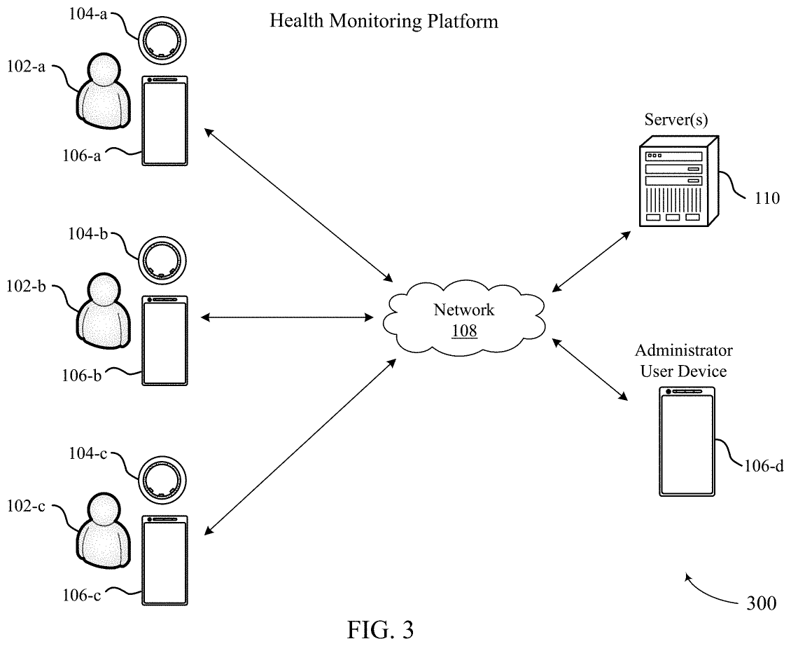

FIG. 3 illustrates an example of a health monitoring platform 300 that supports an anonymized health monitoring platform in accordance with aspects of the present disclosure. The health monitoring platform 300 may implement, or be implemented by, aspects of the system 100, system 200, or both. In particular, health monitoring platform 300 illustrates an example of one or more wearable devices 104, such as rings, and user devices 106, as described with reference to FIGS. 1 and 2. In some examples, health monitoring platform 300 may represent a health monitoring platform for detecting one or more illnesses in one or more users 102.

In some examples, the health monitoring platform 300 may collect data from one or more users 102 via a wearable device 104, such as a ring. Each wearable device 104 may communicate with one or more user devices 106, such as a computer, cellular device, tablet, another wearable device 104, or other user device 106 of a user 102. For example, wearable device 104-a, which may include a ring, may acquire physiological data from the user 102-a and transmit the acquired physiological data to the user device 106-a and/or server 110 for processing. The user device 106-a may then display the processed physiological data and/or other determined parameters/metrics to the user 102-a. Similarly, wearable device 104-b may send data to user device 106-b for display to user 102-b. Wearable device 104-c may send data to user device 106-c for display to user 102-c. In some examples, the health monitoring platform 300 may include any quantity of users 102, such as User 1 through User N (e.g., with wearable device 104-n and user device 106-n). Each user 102 may have any number of wearable devices 104 collecting data, such as sleep data, heart rate data, HRV data, respiratory rate data, temperature data, etc.

The wearable devices 104, user devices 106, or both, may communicate with one or more servers 110 and/or an administrator user device 106-a via a network 108. For example, the wearable devices 104 may communicate directly with the servers 110 via network 108 by transmitting data collected from a user 102 directly to the servers 110 via the network 108. In some other examples, a wearable device 104 may connect with a user device 106 (e.g., via Bluetooth) to send the data collected from a user 102, where the user device 106 may forward the data to the servers 110 via the network 108. For example, wearable device 104-a may collect data from user 102-a (e.g., physiological data) and may send the data to the user device 106-a, where the user device 106-a may forward the data to the server 110 and/or administrator user device 106-d via a network 108. The servers 110 may include databases or data stores (e.g., an application server, a database server, a cloud-based server, a datacenter, or a combination of these or other devices or systems of devices) configured to store received data and/or perform the various processing functions described herein. In this regard, the server 110 may be used for data storage, management, and processing.

In some cases, the users 102 may have access to their own health related data, but others may be unaware of potential illness. For example, a wearable device 104 may collect data that indicates a user 102 may be sick or ill, or otherwise experiencing an immunological response (e.g., an increased respiratory rate, an increased temperature, increased heart rate, or the like relative to baseline data for the user 102). If no preventative measurements are taken by the user 102, the illness may spread to other users 102 interacting with the user 102.

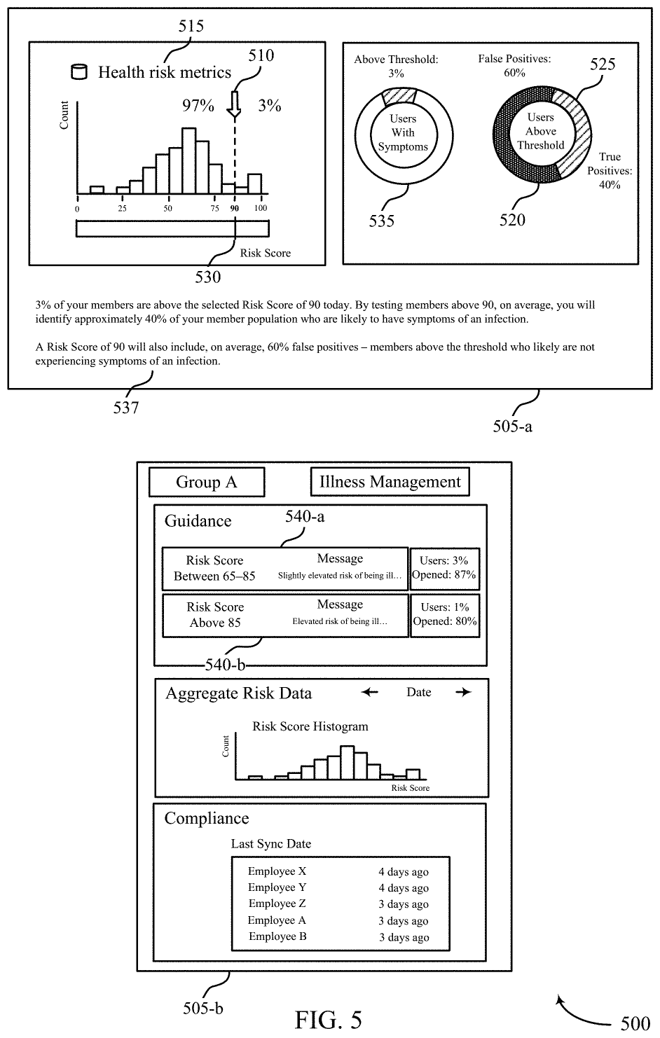

In some examples, the components of the health monitoring platform 300 (e.g., the user device 106-a, the user device 106-b, the user device 106-c, the servers 110, the administrator user device 106-d) may be configured to acquire physiological data from each of the respective users 102, and may calculate scores or metrics associated with each respective user 102. Scores/metrics that may be calculated for each respective user 102 may include Sleep Scores, Readiness Scores, health risk metrics (e.g., illness risk scores), and the like. In some aspects, scores/metrics may be calculated once a day (e.g., once in a 24 hour period), such as when a user 102 accesses the data for the first time each day or at a default time each day. Additionally or alternatively, the health monitoring platform 300 may be configured to transmit determined scores/metrics to the administrator user device 106-a at regular or irregular periodicity (e.g., once a day). Additional details associated with the calculation of metrics/scores for each user 102 (e.g., calculating health risk metrics) are described in further detail with respect to FIG. 5.

In some implementations, physiological data and determined scores/metrics (e.g., health risk metrics) for each respective user may be presented to an administrator (e.g., doctor, health care professional, coach, manager, employer) via the administrator device 106-d in a non-anonymized manner or an anonymized manner. In the context of non-anonymized health monitoring, health information (e.g., physiological data, health risk scores) may be presented via the administrator user device 106-d along with identifiers (e.g., non-anonymized user identifiers) for each respective user. In other words, in the context of non-anonymized health monitoring, the administrator may be able to know which physiological data and health risk metrics correspond to each respective user 102. Comparatively, in the context of anonymized health monitoring, health information (e.g., physiological data, health risk scores) may be presented via the administrator user device 106-d along with anonymized user identifiers for each respective user 102. In other words, in the context of anonymized health monitoring, the administrator may the administrator may not know which physiological data and health risk metrics correspond to each respective user 102. In this regard, anonymized health monitoring techniques described herein may facilitate improved user privacy, and may protect user health information.

In some examples, an administrative personnel operating the administrator user device 106-d (e.g., a user of user device 106-d) may set/adjust a threshold for each category of collected data, for one or more scores calculated from the collected data, or both. For example, an administrator may be able to define (e.g., via the administrator user device 106-d) one or more predefined thresholds for evaluating health risk metrics determined for the respective users 102. In some other examples, the health monitoring platform 300 (e.g., server 110, administrator user device 106-d) may select/determine the one or more predefined thresholds by default, and may communicate the predefined thresholds to the administrator user device 106-d. Additionally or alternatively, the predefined thresholds may be defined (e.g., preconfigured) at the administrator user device 106-d. In some examples, the predefined thresholds may be based on one or more uncertainty rates (e.g., false-positive rates, false-negative rates, true-positive rates, true-negative rates) related to illness detection for the users 102 of the health monitoring platform 300. The selection of predefined thresholds will be described in further detail with respect to FIG. 5.

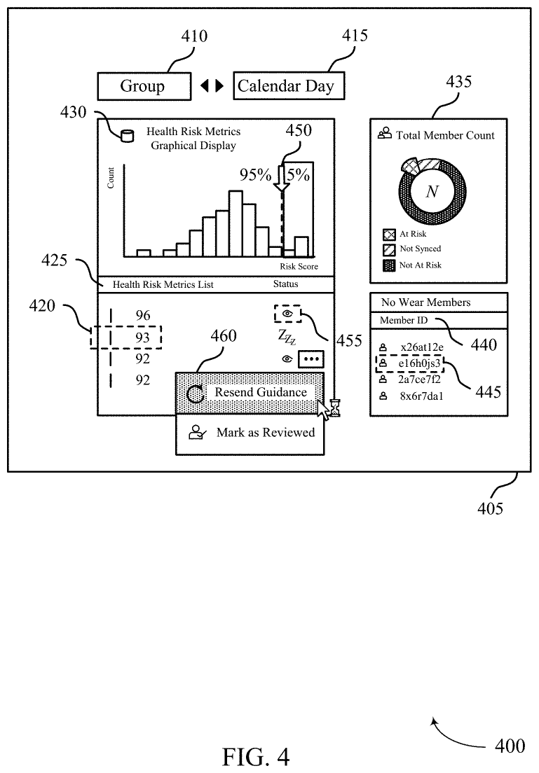

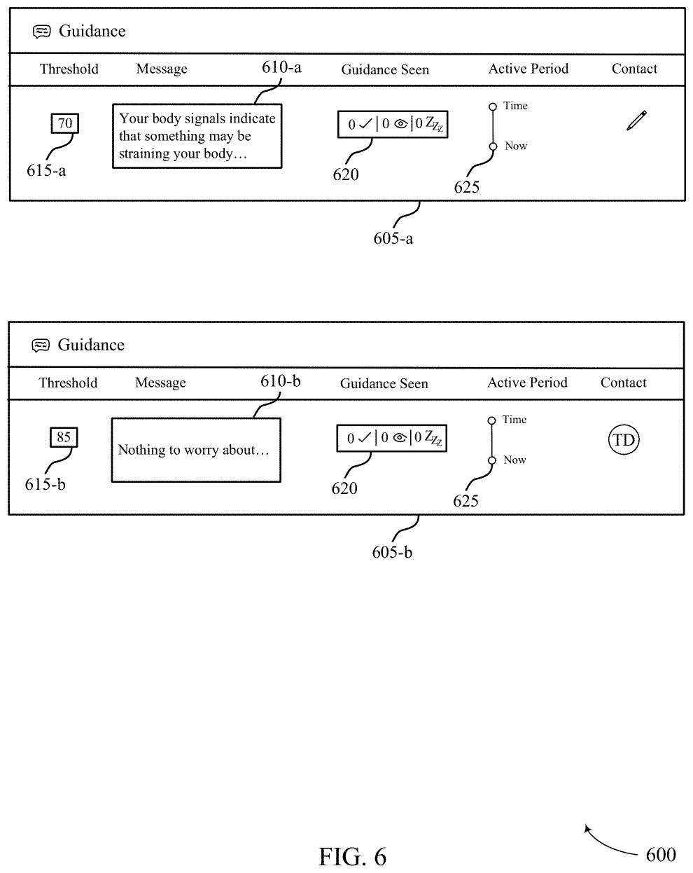

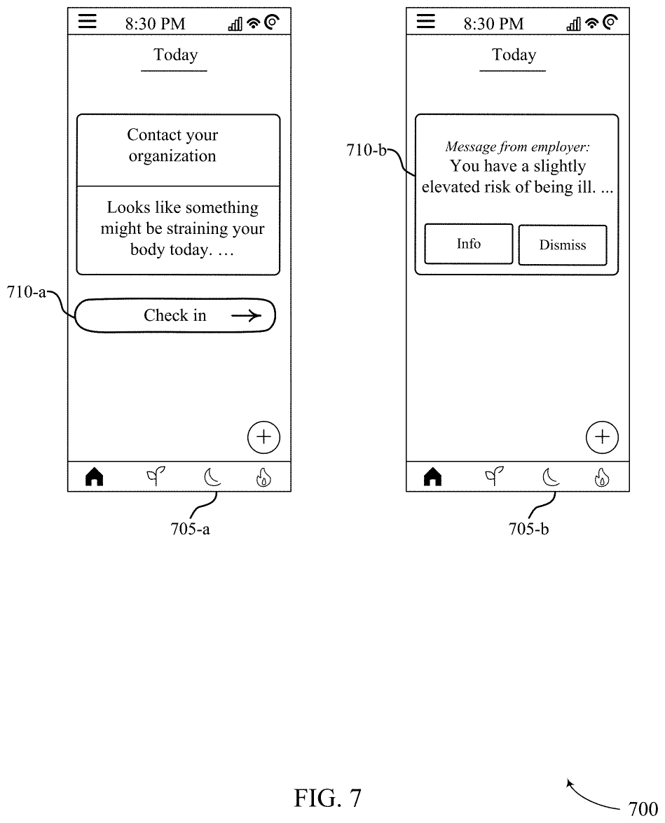

In cases where collected physiological data and/or determined scores/metrics (e.g., health risk metrics) for a user 102 of the health monitoring platform 300 satisfy one or more predefined thresholds, and thereby indicate one or more potential health risks, the health monitoring platform 300 (e.g., server 110, administrator user device 106-d) may transmit messages or notifications to the user 102 associated with each of the one or more potential health risks. For example, if user 102-b exhibits a health risk metric above a predefined threshold, the server 110 may send a message to user device 106-b (e.g., via electronic communication, a push notification to an application running at user device 106-b, or the like). Notifications of potential health risks that may be transmitted to the respective user device 106-a, the user device 106-b, and the user device 106-c, as will be further shown and described with respect to FIG. 7. In some cases, messages transmitted to the user device 106-a, the user device 106-b, and the user device 106-c may be customizable (e.g., via the administrator user device 106-d), which will be described in further detail with respect to FIG. 6. Moreover, an administrator associated with the administrator user device 106-d may track whether respective users 102 have viewed messages related to potential health risks, which will be described in further detail with respect to FIG. 4.

FIG. 4 illustrates an example of a GUI 400 that supports an anonymized health monitoring platform in accordance with aspects of the present disclosure. The GUI 400 may implement, or be implemented by, aspects of the system 100, system 200, health monitoring platform 300, or any combination thereof. For example, the GUI 400 may be an example of a GUI of an administrator user device, such as the administrator user device 106-d of the health monitoring platform 300 as described with reference to FIG. 3.

The GUI 400 illustrates an application page 405, that may be displayed to an administrator/user via the GUI 400 (e.g., GUI of the administrator user device 106-d). Continuing with the example above, an administrator associated with a group 410 of users may check collected data or calculated scores for one or more users in the group 410. The group 410 may be based on a physical location of one or more users (e.g., a floor in an office building), one or more lifestyle parameters for each user (e.g., users with known health risks), employer or organization, or the like. The administrative personnel may be presented with the application page 405 upon opening a platform (e.g., “app”) for viewing collected data from wearable devices 104 (e.g., rings), calculated scores based on the collected data, or both, for the group 410. In some examples, the data may be collected and the scores may be calculated periodically, such as once per calendar day 415 (e.g., every 24 hour period). A system, such as systems as described with reference to FIGS. 1 through 3, may automatically generate a health risk metric 420, such as a risk score, for at least a subset of users in the group 410 when the wearable device syncs collected data.

As shown in FIG. 4, the application page 405 may display one or more health risk metrics 420 for each user. As it is used herein, the terms “health risk metric,” and like terms, may refer to scores or other metrics that indicate a relative probability or likelihood that a given user may experience some health risk (e.g., probability that a user is or will become ill, relative probability that the user will experience a cardiac episode). The health risk metrics 420 may include a value between 0 and 100 (e.g., 93), which a processor of a device, such as a user device, may calculate according to a machine learning algorithm or classifier. For example, the machine learning algorithm may be trained using a set of training physiological data acquired from healthy patients and confirmed sick patients with one or more categories corresponding to the categories of data collected by the wearable devices of the users (heart rate data, HRV data, temperature data, respiratory rate data, etc.). Each health risk metric 420 may be calculated using the machine learning algorithm according to precision and recall curves using collected data from the wearable device as an input. The application page 405 may display calculated health risk metrics 420 in a list 425 as well as a graphical display 430, which may be a histogram showing the calculated health risk metrics 420 for the users of the group 410 in ranges. For example, the range may be 0 to 100 with a step value of 1 if the health risk metric 420 has a value between 0 and 100. The application page 405 may display the health risk metric 420 for each member in the group 410 with a total member count 435.

In some aspects, the system may calculate and display health risk metrics for multiple illnesses, diseases, or conditions. In this regard, each user may be associated with multiple health risk metrics that indicate a relative probability that the respective user will experience multiple different health conditions. For example, each user may be associated with a first health risk metric associated with the flu, a second health risk metric associated with COVID-19, and a third health risk metric associated with cardiac episodes. Moreover, in some implementations, each health risk may be associated with a different respective threshold used to evaluate the relative health risk metric for each user, trigger alerts, and the like. In some cases, the application pages may display the health risk metrics for different health conditions on the same page, or may enable users to “toggle” between application pages associated with different health conditions.

In some examples, the administrative personnel may view a member identifier (ID) 440 for each user in the group 410 (e.g., a user identity or an anonymized user identifier for each respective user). If the member ID 440 includes identity information of the user, the administrative personnel may have access to health data of the users (e.g., non-anonymized health monitoring). In other words, in the context of non-anonymized health monitoring, health risk metrics 420, Sleep/Readiness Scores, and/or acquired physiological data may be presented to an administrator along with non-anonymized user identifiers such that the administrator may determine which users correspond to the respective metrics/scores, acquired data, and the like. However, if the administrative personnel is an employer, sharing health data may compromise privacy and ethical policies (e.g., by asking a user to share potentially personal information).

Thus, as illustrated in the application page 405, the administrative personnel may define messaging rules to codify illness protocol for anonymously contacting at-risk users. In other words, physiological data and health risk metrics may be associated with anonymized user identifiers corresponding to respective users such that administrator personnel are not able to determine which users are associated with determined health risk metrics, acquired physiological data, and the like (e.g., anonymized health monitoring). Using anonymized user identifiers may help protect personal privacy for the respective users, which may be utilized in the context of employer-employee relationships (e.g., user-employees and administrator-employers). With this approach, the administrative personnel may have access to aggregated health and compliance data, but not personal identifiable information for individual users, which may reduce privacy and security concerns, and reduce the amount of manual work involved in directly contacting potential at-risk users.

In some examples, one or more users may opt-in to the group 410 by using a wearable device that collects health related data (e.g., physiological data, or biometrics). In other cases, users may opt-in to the group 410 for health tracking by inputting a user input (e.g., command) into a user device 106 associated with the respective user. For example, the users 102 of the health monitoring platform may be able to opt-in to the health monitoring platform (e.g., opt-in to the group 410) for health tracking by inputting one or more opt-in commands via the user devices 106 associated with each of the respective users. In some implementations, physiological data may not be collected and scores/metrics may not be calculated for a given user until the user has opted-into the health monitoring platform.

In some examples, an administrative personnel (e.g., employer administrators) may access a dashboard, illustrated by the application page 405, which illustrates an anonymized list of users arranged by health risk metric 420 for the respective users of the group 410 for each calendar day 415. For example, the health risk metrics 420 in the list 425 may be sorted from a largest health risk metric 420 (e.g., 96) to a smallest health risk metric 420. A member ID 440 may include a unique code for each user but may not include information related to the identity of the user. For example, as described previously herein, member IDs 440 for the users within the group 410 may include anonymized user identifiers associated with each respective user.

In some cases, the administrative personnel may select a user icon 445 associated with a respective user in order to view more details regarding the user's physiological data and/or determined scores/metrics. For example, by selecting the user icon 445, an administrator may be able to view the user's health risk metrics 420 (e.g., via a line chart) for some historical time period (e.g., the last two weeks). Illustrating the user's historical health risk metrics 420 may enable the administrator to gauge the “severity” of the user's health risk metrics 420. In particular, historical health risk metrics 420 may enable the administrator to determine whether a health risk metric 420 for the user is a random one-off metric, or if the user has exhibited a trend of increasing health risk metrics. In some implementations, additional health data that is displayed upon selection of the user icon 445 may be presented anonymously (e.g., in association with an anonymized user identifier for the respective user) such that the user's identity remains anonymous. In other cases, the additional health data that is displayed upon selection of the user icon 445 may be presented non-anonymously (e.g., in association with a non-anonymized user identifier for the respective user) such that the administrator may determine to which user the data corresponds.

In some examples, administrative personnel may receive a notification (e.g., via electronic communication) if a user in the group 410 exhibits a health risk metric 420 that satisfies one or more thresholds 450 (e.g., predefined thresholds 450). As noted previously herein, the system 200 may utilize or implement different thresholds 450 for different illnesses or health risks, and may calculate different health risk metrics 420 for the different respective illnesses/health risks. The health monitoring platform 300 may contact users that exhibit a health risk metric 420 that satisfies predefined threshold 450 by sending a message to the users. Additionally or alternatively, the health monitoring platform 300 may transmit a notification to the administrator user device 106-d if a user exhibits a health risk metric 420 that satisfies a predefined threshold 450. For example, as shown in the application page 405, the health monitoring platform may determine that eight users exhibit health risk metrics 420 that exceed the predefined threshold 450. In this example, the health monitoring platform 300 may transmit a signal/message to cause the administrator user device 106-d to display a notification of a potential health risk for the eight identified users.

In some implementations, the messages that are transmitted to the user devices 106 of the respective user may be customizable by the administrative personnel (e.g., configurable messages). Users with health risk metrics 420 above the predefined threshold 450 may be referred to as “at-risk users” for a potential health risk, while users with health risk metrics 420 below the predefined threshold 450 may not be at risk. In some cases, the administrative personnel may set the predefined threshold 450, which is described in further detail with respect to FIG. 5. In some other cases, the predefined threshold 450 may be set to a default health risk metric 420, which may be referred to as a baseline threshold (e.g., a score of 90). For example, according to the company-defined protocol, administrators may contact users with a health risk metric 420 above a customer-specific predefined threshold 450 and may inquire about the situation of the user. If the administrator decides there may be a risk that the user is ill, the user may be sent to receive help from a medical professional (e.g., an illness testing kit). The administrative personnel may ask the user to stay away from the rest of the group 410 of users until the illness risk is resolved.

In some examples, upon identifying that a user may exhibit a potential health risk, the health monitoring platform may transmit configurable messages to the user device 106 corresponding to the respective user, which is further illustrated with respect to FIG. 7. In some cases, administrative personnel may be able to see whether the user has viewed or otherwise interacted with (e.g., opened, responded to) the configurable message. For example, a status icon 455 for the configurable message may change once the user views the configurable message. In some cases, the user may acknowledge the configurable message, which may also change the status icon 455. If the user does not acknowledge or view the configurable message, the administrative personnel may resend guidance (e.g., resend the configurable message, or transmit a new configurable message) or may mark the message as viewed via an action menu 460. In other words, the administrator may be able to trigger additional configurable messages that may be sent to the user device 106 of the respective user via the action menu 460.

The application page 405 may display a total member count 435 with a total number of users in a group 410 (e.g., N users in the group 410) as well as percentage of users at risk, not at risk, and without data (e.g., if a wearable device for that user is not synced). For example, the components of the health monitoring platform may identify users who have not worn their wearable devices 104 for some time interval that satisfies a threshold time interval (e.g., users who have not worn their wearable devices 104 for two days). In this example, the health monitoring platform may cause the administrator user device to display an indication of the users who have not worn their wearable devices (e.g., “No Wear Members”). In some cases, the health monitoring platform may display non-anonymized user identifiers for each of the “No Wear Members” so that the administrator may contact the respective users to see why they are not wearing their wearable devices 104, or to request that the users wear their respective wearable devices 104. The health monitoring platform may determine that users are not wearing their wearable devices based on physiological data acquired from the respective users (e.g., temperature readings that indicate that the wearable device 104 is not being worn), an absence of physiological data (e.g., the wearable device 104 is on the charger and is not being worn), or both.

FIG. 5 illustrates an example of a GUI 500 that supports an anonymized health monitoring platform in accordance with aspects of the present disclosure. The GUI 500 may implement, or be implemented by, aspects of the system 100, system 200, health monitoring platform 300, GUI 400, or any combination thereof. For example, the GUI 500 may be an example of a GUI of an administrator user device, such as the administrator user device 106-d of the health monitoring platform 300 as described with reference to FIG. 3.

In some examples, the GUI 500 illustrates a series of application pages 505 that may be displayed to an administrator/user via the GUI 500 (e.g., GUI of the administrator user device). An administrative personnel may adjust one or more predefined thresholds 510, such as a health risk metric threshold related to collected data for one or more users in a group. For example, an administrative personnel may set a threshold 510 based on adjusting the threshold 510 according to a sliding scale 530. The health risk metrics 515 may be calculated based on a likelihood or risk of a user to be sick (e.g., a risk score).

In some cases, the predefined threshold 510 may be determined/selected based on one or more uncertainty rates or illness prediction accuracy metrics related to illness detection for a user. Moreover, an application page 505-a may display an indication of one or more illness prediction accuracy metrics associated with the one or more predefined thresholds 510. Illness prediction accuracy metrics may be associated with a false-positive rate (e.g., rate/percentage of users who will incorrectly be identified as being sick), a false-negative rate (e.g., rate/percentage of users who are actually sick who will be incorrectly identified as being healthy), a true-positive rate (e.g., rate/percentage of users who will correctly be identified as being sick), a true-negative rate (e.g., rate/percentage of users who will correctly be identified as being healthy), or any combination thereof. For instance, the application page 505-a may display a false-positive rate 520 and a true-positive rate 525 for the selected predefined threshold 510.

It may be appreciated herein that the illness prediction accuracy metrics (e.g., uncertainty rates) may be a function of the selected predefined threshold 510, and may therefore change as the administrator changes the threshold 510 via the sliding scale 530. For example, if the predefined threshold 510 is decreased, the false-positive rate 520 may increase, as more users may exhibit health risk metrics 515 that satisfy the decreased threshold 510. By way of another example, if the administrative personnel sets the threshold 510 to 90, and 3% of the users or members in the group are above the threshold 510, then the 3% may be users with symptoms 535. Of the users above the threshold 510, there may be an uncertainty related to the true-positive rate 525 and the false-positive rate 520. For example, 40% of the users with health risk metrics 515 above 90 may be true-positive (e.g., may actually be sick), while 60% of the users with health risk metrics 515 above 90 may be false-positive. Additionally or alternatively, the application page 505-a may display an uncertainty value (e.g., as a percentage) of false-negatives and true-negatives for the threshold 510. The application page 505-a may display the uncertainty rate and an information message 537 related to the uncertainty rate, such that an administrative personnel may make an informed decision when setting the threshold 510. For example, the information message 537 may indicate “3% of your members are above the selected Risk Score of 90 today. By testing members above 90, on average, you will identify approximately 40% of your member population who are likely to have symptoms of an infection,” and “A Risk Score of 90 will also include, on average, 60% false positives—members above the threshold who likely are not experiencing symptoms of an infection.”

In some cases, as illustrated in application page 505-b, an administrator may set/select (e.g., via the administrator user device 106-d) multiple thresholds 510 for determined health risk metrics 515. The use of multiple predefined thresholds 510 may be used to classify a group of users into different health risk categories. Moreover, the administrator may be able to set different configurable messages 540 that may be sent to the users who satisfy the respective predefined thresholds 510. For example, a first user may exhibit a health risk metric 515 between 65 and 85, and a second user may exhibit a health risk metric 515 above 85. In this example, the first user may receive first configurable message 540-a, and the second user may receive a second configurable message 540-b that is different from the first configurable message 540-a. An administrative personnel may set each of the predefined thresholds 510 based on (e.g., according to) displayed or calculated uncertainty metrics (e.g., false-positive rate 520, true-positive rate 525). Moreover, the administrative personnel may input the configurable messages 540 corresponding to the respective predefined thresholds 510 via the administrator user device 106-d. These concepts may be further shown and described with reference to FIG. 6.

FIG. 6 illustrates an example of a GUI 600 that supports an anonymized health monitoring platform in accordance with aspects of the present disclosure. The GUI 600 may implement, or be implemented by, aspects of the system 100, system 200, health monitoring platform 300, GUI 400, GUI 500, or any combination thereof. For example, the GUI 600 may be an example of a GUI of an administrator user device, which may be an example of the administrator user device 106-d of the health monitoring platform 300 as described with reference to FIG. 3.

In some examples, the GUI 600 illustrates a series of application pages 605, such as an application page 605-a and an application page 605-b, which may be displayed to the user via the GUI 600 (e.g., GUI of the administrator user device 106-d). An administrator may input and/or modify one or more configurable messages 610 that may be transmitted to a user device 106 corresponding to a user. For example, in cases where a user of a health monitoring platform exhibits a health risk score that satisfies a predefined threshold, the health monitoring platform may transmit a configurable message 610 to the user device, where the user and the user device may be examples of a user 102 and a user device 106 as described with reference to FIGS. 1 through 3.

In some cases, an administrator may set one or more predefined thresholds 615 against which health risk metrics are to be compared. In particular, the administrator may define multiple predefined thresholds 615, and multiple configurable messages 610 that may be transmitted to users upon satisfaction of the respective predefined thresholds 615. For example, the health monitoring platform (e.g., a server 110) may be configured to transmit the configurable message 610-a to a user device corresponding to a user upon satisfaction of the predefined threshold 615-a, and may be configured to transmit the configurable message 610-b upon satisfaction of the predefined threshold 615-b. In other words, the administrator user device may be used to define different configurable messages 610 for each respective predefined threshold 615.

The configurable messages 610 may guide users to follow an illness protocol for a group. For example, a configurable messages 610-a may indicate that a potential health risk has been identified, such as by indicating “your body signals indicate that something may be straining your body . . . ” based on the threshold 615-a. By way of another example, the configurable messages 610-a may provide one or more recommendations to the user, such as a recommendation for the user to schedule a doctor appointment, to stay home from work or other activities, to prepare for a potential illness by resting or hydrating, and the like. In some other examples, an administrative user may configure a configurable message 610-b to indicate to the user “nothing to worry about . . . ” based on the threshold 615-b.

In some examples, the administrator user device 106-d may display a guidance icon 620 that indicates whether users who have received the respective configurable message 610 have viewed, interacted with, snoozed/dismissed, or otherwise interacted with the configurable message 610. The application page 605-a and the application page 605-b may display an active period 625 for the user and/or the configurable message 610-a and the configurable message 610-b, respectively, along with statistics about how many users were affected and acted on the configurable message 610-a and the configurable message 610-b. In some cases, the application pages 605 may display aggregated statistics associated with health risk metrics per group of users.

In some cases, users may be able to input information associated with health risks via the user devices of the health monitoring platform. For example, a user may indicate (via a user device) whether the user took actions (e.g., took a diagnostic test) to confirm a potential health risk, or whether a diagnostic test (e.g., flu test, COVID test) came back positive or negative.

FIG. 7 illustrates an example of a GUI 700 that supports an anonymized health monitoring platform in accordance with aspects of the present disclosure. The GUI 700 may implement, or be implemented by, aspects of the system 100, system 200, health monitoring platform 300, GUI 400, GUI 500, GUI 600, or any combination thereof. For example, the GUI 700 may be an example of a GUI of a user device corresponding to a user, such as a user device 106 and a user 102 as described with reference to FIG. 3.

In some examples, the GUI 700 may illustrate a series of application pages 705, such as an application page 705-a and an application page 705-b, that may be displayed to a user via the GUI 700 of a user device. As noted previously herein, physiological data collected from a user may be used to calculate scores/metrics (e.g., health risk scores, Sleep Scores, Readiness Scores) for the respective user. Calculated scores/metrics may be displayed to the user via a user device corresponding to the user, as shown in the application pages 705.

In cases where a user's health risk metric satisfies one or more predefined thresholds, as described herein, a health monitoring platform, such as a health monitoring platform 300 as described with reference to FIG. 3 (e.g., server 110, administrator user device 106-d), may transmit one or more configurable messages 710 to the user, where the configurable messages may be associated with potential health risks for the user. For example, the user may receive a configurable message 710-a, which may indicate for the user to check in with a contact person associated with a group of users (e.g., administrator of an office). In some other cases, the user may receive configurable message 710-b, which may prompt the user to check in for more information or dismiss the configurable message 710-b. The configurable messages 710 may be configurable/customizable, such that the user may receive different configurable messages 710 based on different health risk scores, as described previously herein. Moreover, an administrator (e.g., administrator associated with the administrator user device 106-d) may customize the configurable messages 710 that may be transmitted to users within the health monitoring platform.

By enabling administrators to define rules for sending configurable messages 710 to users based on their respective health risk metrics, techniques described herein may automate alerts, messages, and other information that may be provided to users without divulging sensitive information to employer personnel or other administrators.

In some cases, health risk metrics for a user may be determined based on one or more physiological parameters (e.g., temperature, heart rate, HRV, respiratory rate) being different than a baseline set of values for each respective user (e.g., baseline temperature data, baseline HRV data). For example, the health monitoring platform may acquire physiological data from a user (e.g., temperature data, respiratory rate data, HRV data) over a time interval (e.g., two weeks) when the user is in a healthy state. The health monitoring platform may generate baseline physiological data for the user (e.g., customized baseline ranges) based on the acquired physiological data. For instance, acquired physiological data may be used to generate baseline temperature data, baseline respiratory rate data, baseline HRV data, and the like. In some aspects, baseline data sets may take into account circadian features. For instance, the user may naturally exhibit fluctuations in temperature readings throughout the day (e.g., throughout a 24 hour circadian cycle), in which case these natural fluctuations are reflected within the baseline temperature data.

Continuing with the same example, the health monitoring platform may continually acquire physiological data from the user, and compare collected parameters to the user's own baseline parameters. For example, if the user's respiratory rate or heart rate is relatively high compared to a baseline respiratory rate or baseline heart rate for the user, the user's health risk metric may be higher, which may indicate that the user is sick, or likely to become sick (e.g., with the flu or related illness). Similarly, if there are one or more trends (e.g., lack of sleep, high blood pressure, low blood pressure, or the like) that may affect general health of a user or may indicate the presence of one or more preexisting conditions, such as sleep apnea, hypertension, atrial fibrillation, diabetes, and more, the user may receive one or more messages to seek medical advice (e.g., one per preexisting condition, illness, or health risk).

In some implementations, the health monitoring platform may indicate one or more contributing factors that contribute to a user's health risk metric to provide additional insight regarding the user's health risk metric. For example, the application pages 705 may indicate one or more physiological parameters (e.g., contributing factors) that resulted in the user's health risk metric, such as increased temperature, increased respiratory rate, increased heart rate, and the like. In other words, the health monitoring platform may be configured to provide some information or other insights regarding determined health risk metrics. Personalized insights may indicate aspects of collected physiological data (e.g., contributing factors within the physiological data) that were used to generate the health risk metrics.

For example, in cases where a user does not experience high temperatures (e.g., no fever), but still exhibits a relatively high health risk metric (e.g., high probability of illness), providing personalized insights may provide additional information that is driving the high health risk metric. Moreover, providing personalized insights may drive greater user engagement. Further, personalized insights may provide answers to questions such as “What is driving my high health risk metric?”, “Why do I have a high health risk metric if my temperature is normal?”, “Why do I sometimes see big changes in my health risk metric (e.g., health resource management (HRM) score) from one day to the next?”, “Why is my readiness score saying one thing, but my health risk metric score is saying something else?”, and “I have asthma; is my score high because of my respiration rate, or is it picking up on something more?”. In this regard, insights may explain contributing factors for the health risk metrics without exposing raw physiological data, user privacy, or details of the algorithms/models used to generate the health risk metrics.

In some implementations, the health monitoring platform may be configured to receive user inputs regarding detected/predicted illness in order to train classifiers (e.g., supervised learning for a machine learning classifier) and improve illness detection techniques. For example, the user device 106 may display a health risk metric indicating a relative likelihood that the user will become ill. Subsequently, the user may input one or more user inputs, such as an onset of symptoms, a positive illness test, and the like. These user inputs may then be input into the classifier to train the classifier. In other words, the user inputs may be used to validate, or confirm, the determined illness risk metrics.

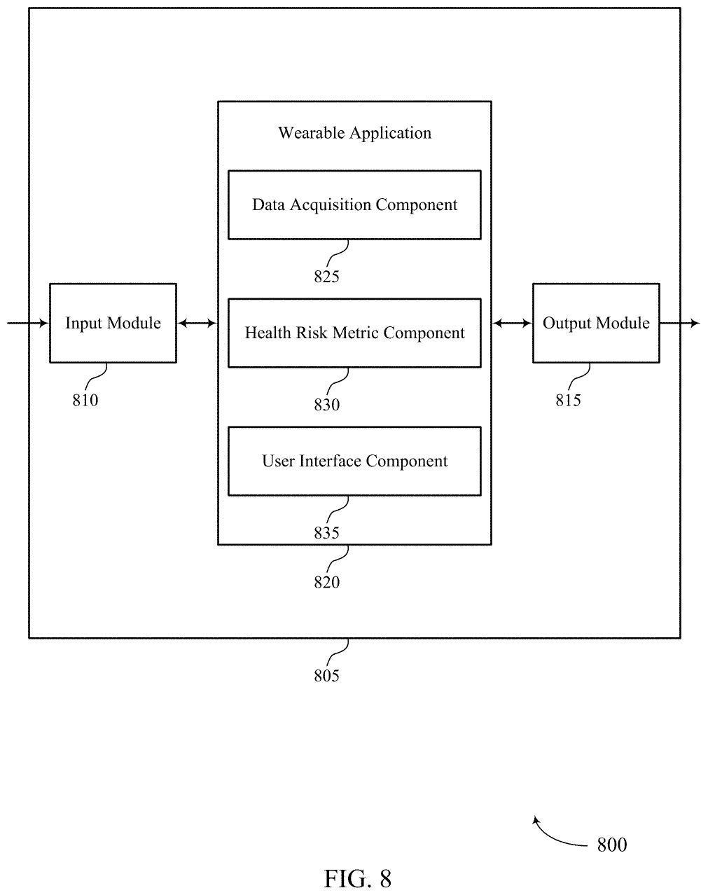

FIG. 8 shows a block diagram 800 of a device 805 that supports an anonymized health monitoring platform in accordance with aspects of the present disclosure. The device 805 may include an input module 810, an output module 815, and a wearable application 820. The device 805 may also include a processor. Each of these components may be in communication with one another (e.g., via one or more buses).

The input module 810 may provide a means for receiving information such as packets, user data, control information, or any combination thereof associated with various information channels (e.g., control channels, data channels, information channels related to illness detection techniques). Information may be passed on to other components of the device 805. The input module 810 may utilize a single antenna or a set of multiple antennas.

The output module 815 may provide a means for transmitting signals generated by other components of the device 805. For example, the output module 815 may transmit information such as packets, user data, control information, or any combination thereof associated with various information channels (e.g., control channels, data channels, information channels related to illness detection techniques). In some examples, the output module 815 may be co-located with an input module 810 in a transceiver module. The output module 815 may utilize a single antenna or a set of multiple antennas.

For example, the wearable application 820 may include a data acquisition component 825, a health risk metric component 830, a user interface component 835, or any combination thereof. In some examples, the wearable application 820, or various components thereof, may be configured to perform various operations (e.g., receiving, monitoring, transmitting) using or otherwise in cooperation with the input module 810, the output module 815, or both. For example, the wearable application 820 may receive information from the input module 810, send information to the output module 815, or be integrated in combination with the input module 810, the output module 815, or both to receive information, transmit information, or perform various other operations as described herein.

The wearable application 820 may support health monitoring techniques in accordance with examples as disclosed herein. The data acquisition component 825 may be configured as or otherwise support a means for receiving physiological data associated with one or more users, the physiological data being continuously collected via one or more wearable devices associated with the respective one or more users, wherein the one or more users are associated with respective anonymized user identifiers. The health risk metric component 830 may be configured as or otherwise support a means for identifying one or more health risk metrics associated with each respective user of the one or more users based at least in part on a respective subset of the physiological data associated with each respective user. The health risk metric component 830 may be configured as or otherwise support a means for identifying a potential health risk for at least one anonymized user identifier corresponding to at least one user based at least in part on the one or more health risk metrics associated with the at least one user satisfying one or more predefined thresholds. The user interface component 835 may be configured as or otherwise support a means for causing a GUI of an administrator user device to display a notification of the potential health risk associated with the at least one anonymized user identifier. The user interface component 835 may be configured as or otherwise support a means for causing an additional GUI of an additional user device corresponding to the at least one user to display a configurable message associated with the potential health risk.

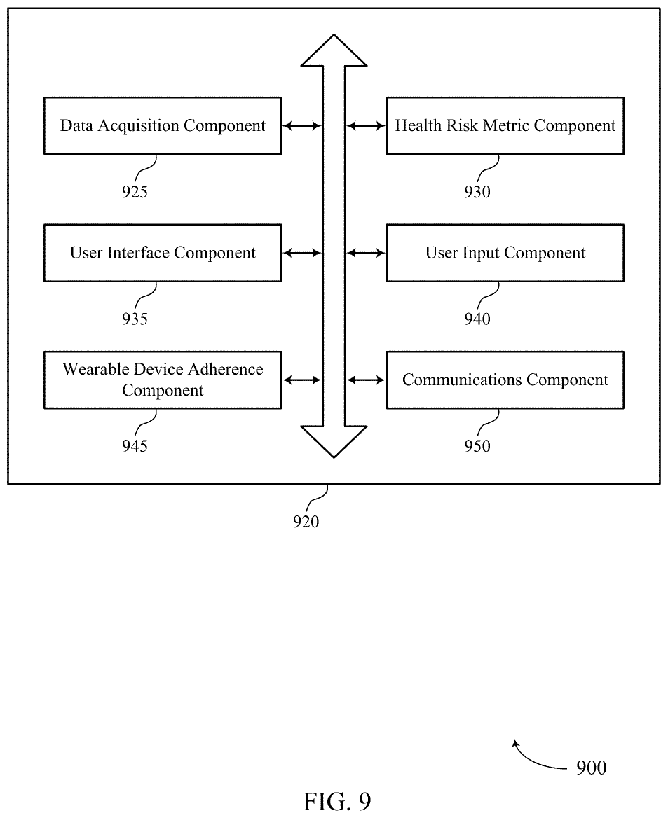

FIG. 9 shows a block diagram 900 of a wearable application 920 that supports an anonymized health monitoring platform in accordance with aspects of the present disclosure. The wearable application 920 may be an example of aspects of a wearable application or a wearable application 820, or both, as described herein. The wearable application 920, or various components thereof, may be an example of means for performing various aspects of an anonymized health monitoring platform as described herein. For example, the wearable application 920 may include a data acquisition component 925, a health risk metric component 930, a user interface component 935, a user input component 940, a wearable device adherence component 945, a communications component 950, or any combination thereof. Each of these components may communicate, directly or indirectly, with one another (e.g., via one or more buses).

The wearable application 920 may support health monitoring techniques in accordance with examples as disclosed herein. The data acquisition component 925 may be configured as or otherwise support a means for receiving physiological data associated with one or more users, the physiological data being continuously collected via one or more wearable devices associated with the respective one or more users, wherein the one or more users are associated with respective anonymized user identifiers. The health risk metric component 930 may be configured as or otherwise support a means for identifying one or more health risk metrics associated with each respective user of the one or more users based at least in part on a respective subset of the physiological data associated with each respective user. In some examples, the health risk metric component 930 may be configured as or otherwise support a means for identifying a potential health risk for at least one anonymized user identifier corresponding to at least one user based at least in part on the one or more health risk metrics associated with the at least one user satisfying one or more predefined thresholds. The user interface component 935 may be configured as or otherwise support a means for causing a GUI of an administrator user device to display a notification of the potential health risk associated with the at least one anonymized user identifier. In some examples, the user interface component 935 may be configured as or otherwise support a means for causing an additional GUI of an additional user device corresponding to the at least one user to display a configurable message associated with the potential health risk.

In some examples, the user input component 940 may be configured as or otherwise support a means for receiving, via the administrator user device, a user input comprising the configurable message, wherein causing the additional user device to display the configurable message is based at least in part on receiving the user input.

In some examples, the user input component 940 may be configured as or otherwise support a means for receiving, via the administrator user device, a user input comprising the one or more predefined thresholds, wherein identifying the potential health risk for the at least one anonymized user identifier is based at least in part on receiving the user input.

In some examples, the user input component 940 may be configured as or otherwise support a means for receiving, via the administrator user device, a first user input comprising a first predefined threshold and a second predefined threshold, the first and second predefined thresholds being included within the one or more predefined thresholds. In some examples, the user input component 940 may be configured as or otherwise support a means for receiving, via the administrator user device, a second user input comprising a first configurable message associated with satisfaction of the first predefined threshold and a second configurable message associated with satisfaction of the second predefined threshold.

In some examples, to support causing the additional user device to display the configurable message, the user interface component 935 may be configured as or otherwise support a means for causing the additional GUI of the additional user device to display the first configurable message based at least in part on identifying that the one or more health risk metrics associated with the at least one user satisfy the first predefined threshold. In some examples, to support causing the additional user device to display the configurable message, the user interface component 935 may be configured as or otherwise support a means for causing the additional GUI of the additional user device to display the second configurable message based at least in part on identifying that the one or more health risk metrics associated with the at least one user satisfy the second predefined threshold.

In some examples, the user interface component 935 may be configured as or otherwise support a means for causing the GUI of the administrator user device to display an indication of one or more illness prediction accuracy metrics associated with the one or more predefined thresholds, the one or more illness prediction accuracy metrics associated with a false-positive rate for the one or more predefined thresholds, a true-positive rate for the one or more predefined thresholds, a false-negative rate for the one more predefined thresholds, a true-negative rate for the one or more predefined thresholds, or any combination thereof.

In some examples, to support causing the administrator device to display the notification, the user interface component 935 may be configured as or otherwise support a means for causing the GUI of the administrator user device to display an indication of the at least one anonymized user identifier and the one or more health risk metrics associated with the user corresponding to the at least one anonymized user identifier.

In some examples, the user interface component 935 may be configured as or otherwise support a means for causing the GUI of the administrator user device to display an indication that the configurable message has been provided to the additional user device corresponding to the at least one user.

In some examples, the user interface component 935 may be configured as or otherwise support a means for causing the GUI of the administrator user device to display an indication that the at least one user has not viewed or interacted with the configurable message.

In some examples, the user interface component 935 may be configured as or otherwise support a means for causing the GUI of the additional user device to display a second configurable message associated with the potential health risk based at least in part on the indication that the at least one user has not viewed or interacted with the configurable message.

In some examples, the user input component 940 may be configured as or otherwise support a means for receiving, via the administrator user device, a user input to trigger the second configurable message, wherein causing the GUI of the additional user device to display the second configurable message is based at least in part on receiving the user input.

In some examples, the wearable device adherence component 945 may be configured as or otherwise support a means for identifying a second user of the one or more users who has not worn the respective wearable device associated with the second user for a time interval that satisfies a threshold time interval. In some examples, the user interface component 935 may be configured as or otherwise support a means for causing a GUI of the administrator user device to display a non-anonymized user identifier associated with the second user based at least in part on identifying that the second user has not worn the respective wearable device for the time interval.

In some examples, identifying the second user of the one or more users who has not worn the respective wearable device is based at least in part on a portion of the physiological data received from the respective wearable device associated with the second user, an absence of physiological data received from the respective wearable device associated with the second user, or both.

In some examples, the user input component 940 may be configured as or otherwise support a means for receiving one or more opt-in commands from the one or more user devices associated with the respective one or more users, wherein receiving the physiological data associated with the one or more users is based at least in part on receiving the one or more opt-in commands.

In some examples, the health risk metric component 930 may be configured as or otherwise support a means for identifying one or more contributing factors for the potential health risk for the at least one anonymized user identifier. In some examples, the user interface component 935 may be configured as or otherwise support a means for causing the GUI of the administrator user device, the additional GUI of the additional user device, or both, to display an indication of the one or more contributing factors.

In some examples, to support causing the administrator user device to display the notification of the potential health risk associated with the at least one anonymized user identifier, the communications component 950 may be configured as or otherwise support a means for transmitting the notification to the administrator user device, wherein the notification comprises an email, a text message, a push notification, or any combination thereof.

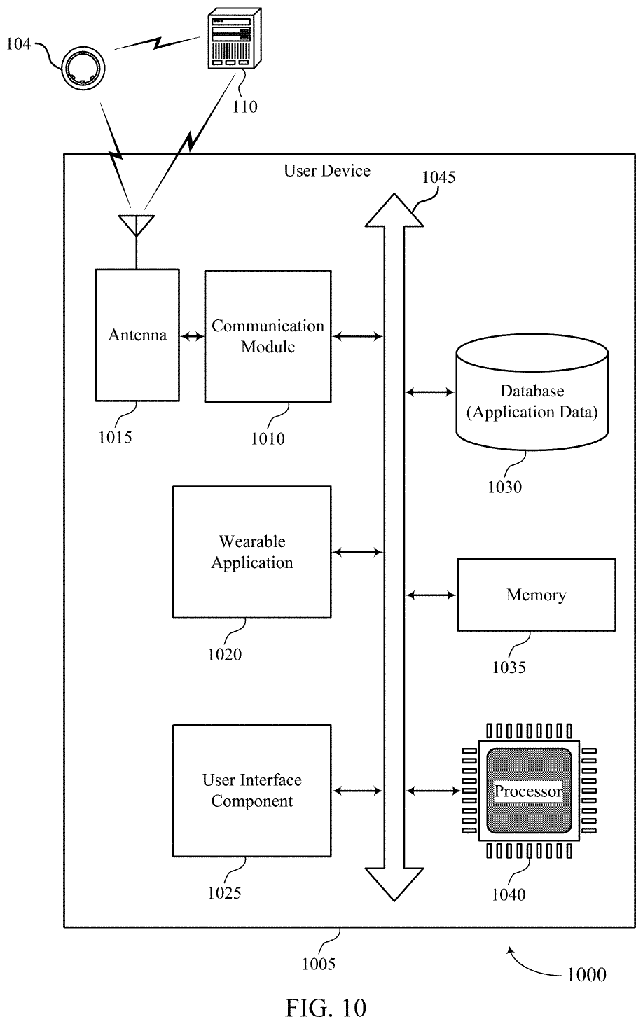

FIG. 10 shows a diagram of a system 1000 including a device 1005 that supports an anonymized health monitoring platform in accordance with aspects of the present disclosure. The device 1005 may be an example of or include the components of a device 805 as described herein. In some implementations, the device 1005 may include an example of a user device 106 and/or server 110, as described herein. The device 1005 may include components for bi-directional communications with components described herein (e.g., ring 104, server 110, user device 106), such as a wearable application 1020, a communication module 1010, an antenna 1015, a user interface component 1025, a database (application data) 1030, a memory 1035, and a processor 1040. These components may be in electronic communication or otherwise coupled (e.g., operatively, communicatively, functionally, electronically, electrically) via one or more buses (e.g., a bus 1045).

The communication module 1010 may manage input and output signals for the device 1005 via the antenna 1015. The communication module 1010 may include an example of the communication module 220-b of the user device 106 shown and described in FIG. 2. In this regard, the communication module 1010 may manage communications with the ring 104 and the server 110, as illustrated in FIG. 2. The communication module 1010 may also manage peripherals not integrated into the device 1005. In some cases, the communication module 1010 may represent a physical connection or port to an external peripheral. In some cases, the communication module 1010 may utilize an operating system such as iOS®, ANDROID®, MS-DOS®, MS-WINDOWS®, OS/2®, UNIX®, LINUX®, or another known operating system. In other cases, the communication module 1010 may represent or interact with a wearable device (e.g., ring 104), modem, a keyboard, a mouse, a touchscreen, or a similar device. In some cases, the communication module 1010 may be implemented as part of the processor 1040. In some examples, a user may interact with the device 1005 via the communication module 1010, user interface component 1025, or via hardware components controlled by the communication module 1010.

In some cases, the device 1005 may include a single antenna 1015. However, in some other cases, the device 1005 may have more than one antenna 1015, which may be capable of concurrently transmitting or receiving multiple wireless transmissions. The communication module 1010 may communicate bi-directionally, via the one or more antennas 1015, wired, or wireless links as described herein. For example, the communication module 1010 may represent a wireless transceiver and may communicate bi-directionally with another wireless transceiver. The communication module 1010 may also include a modem to modulate the packets, to provide the modulated packets to one or more antennas 1015 for transmission, and to demodulate packets received from the one or more antennas 1015.

The user interface component 1025 may manage data storage and processing in a database 1030. In some cases, a user may interact with the user interface component 1025. In other cases, the user interface component 1025 may operate automatically without user interaction. The database 1030 may be an example of a single database, a distributed database, multiple distributed databases, a data store, a data lake, or an emergency backup database.

The memory 1035 may include RAM and ROM. The memory 1035 may store computer-readable, computer-executable software including instructions that, when executed, cause the processor 1040 to perform various functions described herein. In some cases, the memory 1035 may contain, among other things, a basic I/O system (BIOS) which may control basic hardware or software operation such as the interaction with peripheral components or devices.

The processor 1040 may include an intelligent hardware device, (e.g., a general-purpose processor, a digital signal processor (DSP), a central processing unit (CPU), a microcontroller, an application-specific integrated circuit (ASIC), a field-programmable gate array (FPGA), a programmable logic device, a discrete gate or transistor logic component, a discrete hardware component, or any combination thereof). In some cases, the processor 1040 may be configured to operate a memory array using a memory controller. In other cases, a memory controller may be integrated into the processor 1040. The processor 1040 may be configured to execute computer-readable instructions stored in a memory 1035 to perform various functions (e.g., functions or tasks supporting a method and system for sleep staging algorithms).

The wearable application 1020 may support health monitoring techniques in accordance with examples as disclosed herein. For example, the wearable application 1020 may be configured as or otherwise support a means for receiving physiological data associated with one or more users, the physiological data being continuously collected via one or more wearable devices associated with the respective one or more users, wherein the one or more users are associated with respective anonymized user identifiers. The wearable application 1020 may be configured as or otherwise support a means for identifying one or more health risk metrics associated with each respective user of the one or more users based at least in part on a respective subset of the physiological data associated with each respective user. The wearable application 1020 may be configured as or otherwise support a means for identifying a potential health risk for at least one anonymized user identifier corresponding to at least one user based at least in part on the one or more health risk metrics associated with the at least one user satisfying one or more predefined thresholds. The wearable application 1020 may be configured as or otherwise support a means for causing a GUI of an administrator user device to display a notification of the potential health risk associated with the at least one anonymized user identifier. The wearable application 1020 may be configured as or otherwise support a means for causing an additional GUI of an additional user device corresponding to the at least one user to display a configurable message associated with the potential health risk.

By including or configuring the wearable application 1020 in accordance with examples as described herein, the device 1005 may support techniques for improved illness detection/prediction. In particular, techniques described herein may enable a health monitoring platform that is configured to alert users and/or administrators that a user may transition from a healthy state to an unhealthy state. As such, techniques described herein may reduce a spread of illness, and reduce a severity of illness. Moreover, techniques described herein may enable anonymized health monitoring, which may improve user privacy and protect user health data.

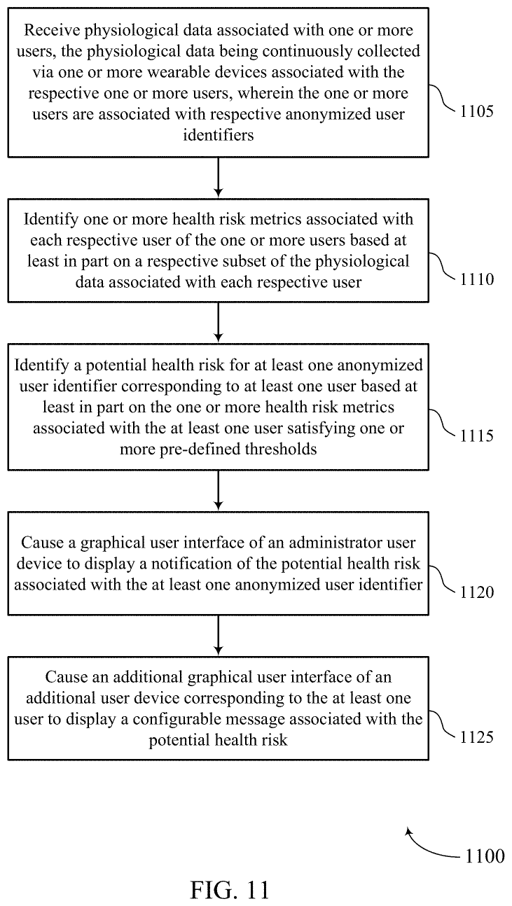

FIG. 11 shows a flowchart illustrating a method 1100 that supports an anonymized health monitoring platform in accordance with aspects of the present disclosure. The operations of the method 1100 may be implemented by a User Device or its components as described herein. For example, the operations of the method 1100 may be performed by a User Device as described with reference to FIGS. 1 through 10. In some examples, a User Device may execute a set of instructions to control the functional elements of the User Device to perform the described functions. Additionally or alternatively, the User Device may perform aspects of the described functions using special-purpose hardware.

At 1105, the method may include receiving physiological data associated with one or more users, the physiological data being continuously collected via one or more wearable devices associated with the respective one or more users, wherein the one or more users are associated with respective anonymized user identifiers. The operations of 1105 may be performed in accordance with examples as disclosed herein. In some examples, aspects of the operations of 1105 may be performed by a data acquisition component 925 as described with reference to FIG. 9.

At 1110, the method may include identifying one or more health risk metrics associated with each respective user of the one or more users based at least in part on a respective subset of the physiological data associated with each respective user. The operations of 1110 may be performed in accordance with examples as disclosed herein. In some examples, aspects of the operations of 1110 may be performed by a health risk metric component 930 as described with reference to FIG. 9.

At 1115, the method may include identifying a potential health risk for at least one anonymized user identifier corresponding to at least one user based at least in part on the one or more health risk metrics associated with the at least one user satisfying one or more predefined thresholds. The operations of 1115 may be performed in accordance with examples as disclosed herein. In some examples, aspects of the operations of 1115 may be performed by a health risk metric component 930 as described with reference to FIG. 9.

At 1120, the method may include causing a GUI of an administrator user device to display a notification of the potential health risk associated with the at least one anonymized user identifier. The operations of 1120 may be performed in accordance with examples as disclosed herein. In some examples, aspects of the operations of 1120 may be performed by a user interface component 935 as described with reference to FIG. 9.

At 1125, the method may include causing an additional GUI of an additional user device corresponding to the at least one user to display a configurable message associated with the potential health risk. The operations of 1125 may be performed in accordance with examples as disclosed herein. In some examples, aspects of the operations of 1125 may be performed by a user interface component 935 as described with reference to FIG. 9.

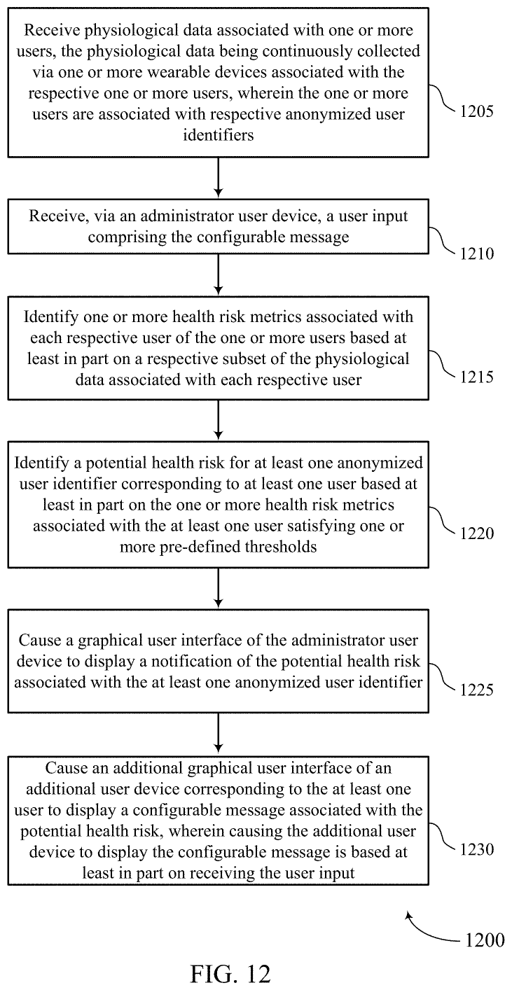

FIG. 12 shows a flowchart illustrating a method 1200 that supports an anonymized health monitoring platform in accordance with aspects of the present disclosure. The operations of the method 1200 may be implemented by a User Device or its components as described herein. For example, the operations of the method 1200 may be performed by a User Device as described with reference to FIGS. 1 through 10. In some examples, a User Device may execute a set of instructions to control the functional elements of the User Device to perform the described functions. Additionally or alternatively, the User Device may perform aspects of the described functions using special-purpose hardware.

At 1205, the method may include receiving physiological data associated with one or more users, the physiological data being continuously collected via one or more wearable devices associated with the respective one or more users, wherein the one or more users are associated with respective anonymized user identifiers. The operations of 1205 may be performed in accordance with examples as disclosed herein. In some examples, aspects of the operations of 1205 may be performed by a data acquisition component 925 as described with reference to FIG. 9.

At 1210, the method may include receiving, via an administrator user device, a user input comprising the configurable message. The operations of 1210 may be performed in accordance with examples as disclosed herein. In some examples, aspects of the operations of 1210 may be performed by a user input component 940 as described with reference to FIG. 9.

At 1215, the method may include identifying one or more health risk metrics associated with each respective user of the one or more users based at least in part on a respective subset of the physiological data associated with each respective user. The operations of 1215 may be performed in accordance with examples as disclosed herein. In some examples, aspects of the operations of 1215 may be performed by a health risk metric component 930 as described with reference to FIG. 9.

At 1220, the method may include identifying a potential health risk for at least one anonymized user identifier corresponding to at least one user based at least in part on the one or more health risk metrics associated with the at least one user satisfying one or more predefined thresholds. The operations of 1220 may be performed in accordance with examples as disclosed herein. In some examples, aspects of the operations of 1220 may be performed by a health risk metric component 930 as described with reference to FIG. 9.

At 1225, the method may include causing a GUI of the administrator user device to display a notification of the potential health risk associated with the at least one anonymized user identifier. The operations of 1225 may be performed in accordance with examples as disclosed herein. In some examples, aspects of the operations of 1225 may be performed by a user interface component 935 as described with reference to FIG. 9.

At 1230, the method may include causing an additional GUI of an additional user device corresponding to the at least one user to display a configurable message associated with the potential health risk. The operations of 1230 may be performed in accordance with examples as disclosed herein. In some examples, aspects of the operations of 1230 may be performed by a user interface component 935 as described with reference to FIG. 9.

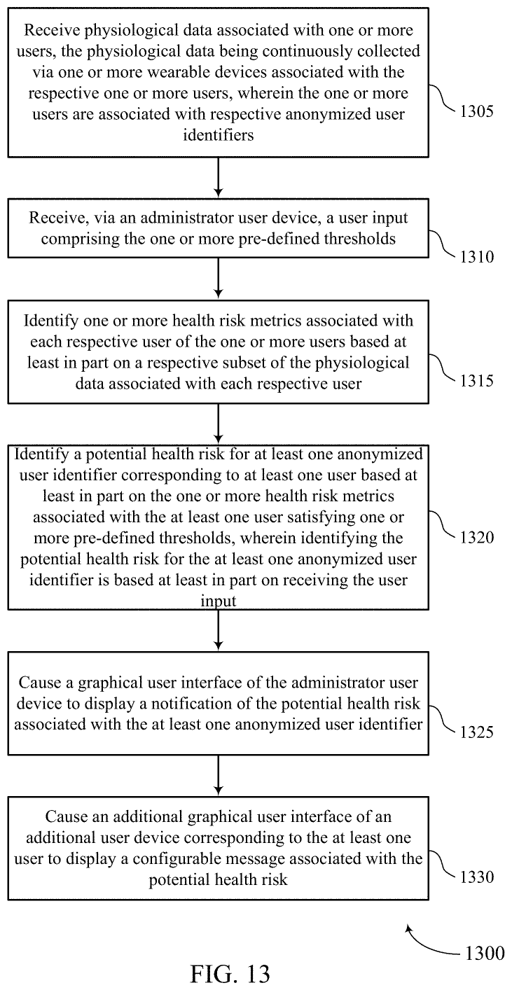

FIG. 13 shows a flowchart illustrating a method 1300 that supports an anonymized health monitoring platform in accordance with aspects of the present disclosure. The operations of the method 1300 may be implemented by a User Device or its components as described herein. For example, the operations of the method 1300 may be performed by a User Device as described with reference to FIGS. 1 through 10. In some examples, a User Device may execute a set of instructions to control the functional elements of the User Device to perform the described functions. Additionally or alternatively, the User Device may perform aspects of the described functions using special-purpose hardware.

At 1305, the method may include receiving physiological data associated with one or more users, the physiological data being continuously collected via one or more wearable devices associated with the respective one or more users, wherein the one or more users are associated with respective anonymized user identifiers. The operations of 1305 may be performed in accordance with examples as disclosed herein. In some examples, aspects of the operations of 1305 may be performed by a data acquisition component 925 as described with reference to FIG. 9.

At 1310, the method may include receiving, via the administrator user device, a user input comprising the one or more predefined thresholds. The operations of 1310 may be performed in accordance with examples as disclosed herein. In some examples, aspects of the operations of 1310 may be performed by a user input component 940 as described with reference to FIG. 9.

At 1315, the method may include identifying one or more health risk metrics associated with each respective user of the one or more users based at least in part on a respective subset of the physiological data associated with each respective user. The operations of 1315 may be performed in accordance with examples as disclosed herein. In some examples, aspects of the operations of 1315 may be performed by a health risk metric component 930 as described with reference to FIG. 9.

At 1320, the method may include identifying a potential health risk for at least one anonymized user identifier corresponding to at least one user based at least in part on the one or more health risk metrics associated with the at least one user satisfying one or more predefined thresholds, wherein identifying the potential health risk for the at least one anonymized user identifier is based at least in part on receiving the user input. The operations of 1320 may be performed in accordance with examples as disclosed herein. In some examples, aspects of the operations of 1320 may be performed by a health risk metric component 930 as described with reference to FIG. 9.

At 1325, the method may include causing a GUI of the administrator user device to display a notification of the potential health risk associated with the at least one anonymized user identifier. The operations of 1325 may be performed in accordance with examples as disclosed herein. In some examples, aspects of the operations of 1325 may be performed by a user interface component 935 as described with reference to FIG. 9.

At 1330, the method may include causing an additional GUI of an additional user device corresponding to the at least one user to display a configurable message associated with the potential health risk. The operations of 1330 may be performed in accordance with examples as disclosed herein. In some examples, aspects of the operations of 1330 may be performed by a user interface component 935 as described with reference to FIG. 9.

The following provides an overview of the present disclosure:

A method for health monitoring is described. The method may include receiving physiological data associated with one or more users, the physiological data being continuously collected via one or more wearable devices associated with each respective user of the one or more users, wherein the one or more users are associated with respective anonymized user identifiers, identifying one or more health risk metrics associated with the each respective user of the one or more users based at least in part on a respective subset of the physiological data associated with the each respective user, identifying a potential health risk for at least one anonymized user identifier corresponding to at least one user based at least in part on the one or more health risk metrics associated with the at least one user satisfying one or more predefined thresholds, causing a GUI of an administrator user device to display a notification of the potential health risk associated with the at least one anonymized user identifier, and causing an additional GUI of an additional user device corresponding to the at least one user to display a configurable message associated with the potential health risk.

An apparatus for health monitoring is described. The apparatus may include a processor, memory coupled with the processor, and instructions stored in the memory. The instructions may be executable by the processor to cause the apparatus to receive physiological data associated with one or more users, the physiological data being continuously collected via one or more wearable devices associated with each respective user of the one or more users, wherein the one or more users are associated with respective anonymized user identifiers, identify one or more health risk metrics associated with the each respective user of the one or more users based at least in part on a respective subset of the physiological data associated with the each respective user, identify a potential health risk for at least one anonymized user identifier corresponding to at least one user based at least in part on the one or more health risk metrics associated with the at least one user satisfying one or more predefined thresholds, cause a GUI of an administrator user device to display a notification of the potential health risk associated with the at least one anonymized user identifier, and cause an additional GUI of an additional user device corresponding to the at least one user to display a configurable message associated with the potential health risk.

Another apparatus for health monitoring is described. The apparatus may include means for receiving physiological data associated with one or more users, the physiological data being continuously collected via one or more wearable devices associated with each respective user of the one or more users, wherein the one or more users are associated with respective anonymized user identifiers, means for identifying one or more health risk metrics associated with the each respective user of the one or more users based at least in part on a respective subset of the physiological data associated with the each respective user, means for identifying a potential health risk for at least one anonymized user identifier corresponding to at least one user based at least in part on the one or more health risk metrics associated with the at least one user satisfying one or more predefined thresholds, means for causing a GUI of an administrator user device to display a notification of the potential health risk associated with the at least one anonymized user identifier, and means for causing an additional GUI of an additional user device corresponding to the at least one user to display a configurable message associated with the potential health risk.

A non-transitory computer-readable medium storing code for health monitoring is described. The code may include instructions executable by a processor to receive physiological data associated with one or more users, the physiological data being continuously collected via one or more wearable devices associated with each respective user of the one or more users, wherein the one or more users are associated with respective anonymized user identifiers, identify one or more health risk metrics associated with the each respective user of the one or more users based at least in part on a respective subset of the physiological data associated with the each respective user, identify a potential health risk for at least one anonymized user identifier corresponding to at least one user based at least in part on the one or more health risk metrics associated with the at least one user satisfying one or more predefined thresholds, cause a GUI of an administrator user device to display a notification of the potential health risk associated with the at least one anonymized user identifier, and cause an additional GUI of an additional user device corresponding to the at least one user to display a configurable message associated with the potential health risk.

Some examples of the method, apparatuses, and non-transitory computer-readable medium described herein may further include operations, features, means, or instructions for receiving, via the administrator user device, a user input comprising the configurable message, wherein causing the additional user device to display the configurable message may be based at least in part on receiving the user input.

Some examples of the method, apparatuses, and non-transitory computer-readable medium described herein may further include operations, features, means, or instructions for receiving, via the administrator user device, a user input comprising the one or more predefined thresholds, wherein identifying the potential health risk for the at least one anonymized user identifier may be based at least in part on receiving the user input.

Some examples of the method, apparatuses, and non-transitory computer-readable medium described herein may further include operations, features, means, or instructions for receiving, via the administrator user device, a first user input comprising a first predefined threshold and a second predefined threshold, wherein the first predefined threshold and the second predefined threshold may be included within the one or more predefined thresholds and receiving, via the administrator user device, a second user input comprising a first configurable message associated with satisfaction of the first predefined threshold and a second configurable message associated with satisfaction of the second predefined threshold.

In some examples of the method, apparatuses, and non-transitory computer-readable medium described herein, causing the additional user device to display the configurable message may include operations, features, means, or instructions for causing the additional GUI of the additional user device to display the first configurable message based at least in part on identifying that the one or more health risk metrics associated with the at least one user satisfy the first predefined threshold and causing the additional GUI of the additional user device to display the second configurable message based at least in part on identifying that the one or more health risk metrics associated with the at least one user satisfy the second predefined threshold.

Some examples of the method, apparatuses, and non-transitory computer-readable medium described herein may further include operations, features, means, or instructions for causing the GUI of the administrator user device to display an indication of one or more illness prediction accuracy metrics associated with the one or more predefined thresholds, the one or more illness prediction accuracy metrics associated with a false-positive rate for the one or more predefined thresholds, a true-positive rate for the one or more predefined thresholds, a false-negative rate for the one or more predefined thresholds, a true-negative rate for the one or more predefined thresholds, or any combination thereof.

In some examples of the method, apparatuses, and non-transitory computer-readable medium described herein, causing the administrator user device to display the notification may include operations, features, means, or instructions for causing the GUI of the administrator user device to display an indication of the at least one anonymized user identifier and the one or more health risk metrics associated with a user corresponding to the at least one anonymized user identifier.

Some examples of the method, apparatuses, and non-transitory computer-readable medium described herein may further include operations, features, means, or instructions for causing the GUI of the administrator user device to display an indication that the configurable message may have been provided to the additional user device corresponding to the at least one user.

Some examples of the method, apparatuses, and non-transitory computer-readable medium described herein may further include operations, features, means, or instructions for causing the GUI of the administrator user device to display an indication that the at least one user may have not viewed or interacted with the configurable message.

Some examples of the method, apparatuses, and non-transitory computer-readable medium described herein may further include operations, features, means, or instructions for causing the GUI of the additional user device to display a second configurable message associated with the potential health risk based at least in part on the indication that the at least one user may have not viewed or interacted with the configurable message.

Some examples of the method, apparatuses, and non-transitory computer-readable medium described herein may further include operations, features, means, or instructions for receiving, via the administrator user device, a user input to trigger the second configurable message, wherein causing the GUI of the additional user device to display the second configurable message may be based at least in part on receiving the user input.

Some examples of the method, apparatuses, and non-transitory computer-readable medium described herein may further include operations, features, means, or instructions for identifying a second user of the one or more users who may have not worn a respective wearable device associated with the second user for a time interval that satisfies a threshold time interval and causing the GUI of the administrator user device to display a non-anonymized user identifier associated with the second user based at least in part on identifying that the second user may have not worn the respective wearable device for the time interval.

Some examples of the method, apparatuses, and non-transitory computer-readable medium described herein may further include operations, features, means, or instructions for identifying the second user of the one or more users who may have not worn the respective wearable device may be based at least in part on a portion of the physiological data received from the respective wearable device associated with the second user, an absence of physiological data received from the respective wearable device associated with the second user, or both.

Some examples of the method, apparatuses, and non-transitory computer-readable medium described herein may further include operations, features, means, or instructions for receiving one or more opt-in commands from one or more user devices associated with respective one or more users, wherein receiving the physiological data associated with the one or more users may be based at least in part on receiving the one or more opt-in commands.

Some examples of the method, apparatuses, and non-transitory computer-readable medium described herein may further include operations, features, means, or instructions for identifying one or more contributing factors for the potential health risk for the at least one anonymized user identifier and causing the GUI of the administrator user device, the additional GUI of the additional user device, or both, to display an indication of the one or more contributing factors.

In some examples of the method, apparatuses, and non-transitory computer-readable medium described herein, causing the administrator user device to display the notification of the potential health risk associated with the at least one anonymized user identifier may include operations, features, means, or instructions for transmitting the notification to the administrator user device, wherein the notification comprises an email, a text message, a push notification, or any combination thereof.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for health monitoring, comprising:
receiving physiological data associated with one or more users, the physiological data being continuously collected via one or more wearable devices associated with each respective user of the one or more users, wherein the one or more users are associated with respective anonymized user identifiers;
identifying one or more health risk metrics associated with each respective user of the one or more users based at least in part on a respective subset of the physiological data associated with each respective user; and
identifying a potential health risk for at least one anonymized user identifier corresponding to at least one user based at least in part on the one or more health risk metrics associated with the at least one user satisfying one or more predefined thresholds.

2. The method of claim 1, further comprising:
causing a graphical user interface of an administrator user device to display a notification of the potential health risk associated with the at least one anonymized user identifier; and
causing an additional graphical user interface of an additional user device corresponding to the at least one user to display a configurable message associated with the potential health risk.

3. The method of claim 2, further comprising:
receiving, via the administrator user device, a user input comprising the configurable message, wherein causing the additional user device to display the configurable message is based at least in part on receiving the user input.

4. The method of claim 2, further comprising:
receiving, via the administrator user device, a user input comprising the one or more predefined thresholds, wherein identifying the potential health risk for the at least one anonymized user identifier is based at least in part on receiving the user input.

5. The method of claim 2, further comprising:
receiving, via the administrator user device, a first user input comprising a first predefined threshold and a second predefined threshold, wherein the first predefined threshold and the second predefined threshold are included within the one or more predefined thresholds; and
receiving, via the administrator user device, a second user input comprising a first configurable message associated with satisfaction of the first predefined threshold and a second configurable message associated with satisfaction of the second predefined threshold.

6. The method of claim 5, wherein causing the additional user device to display the configurable message comprises:
causing the additional graphical user interface of the additional user device to display the first configurable message based at least in part on identifying that the one or more health risk metrics associated with the at least one user satisfy the first predefined threshold; and
causing the additional graphical user interface of the additional user device to display the second configurable message based at least in part on identifying that the one or more health risk metrics associated with the at least one user satisfy the second predefined threshold.

7. The method of claim 2, further comprising:
causing the graphical user interface of the administrator user device to display an indication of one or more illness prediction accuracy metrics associated with the one or more predefined thresholds, the one or more illness prediction accuracy metrics associated with a false-positive rate for the one or more predefined thresholds, a true-positive rate for the one or more predefined thresholds, a false-negative rate for the one or more predefined thresholds, a true-negative rate for the one or more predefined thresholds, or any combination thereof.

8. The method of claim 2, wherein causing the administrator user device to display the notification comprises:
causing the graphical user interface of the administrator user device to display an indication of the at least one anonymized user identifier and the one or more health risk metrics associated with a user corresponding to the at least one anonymized user identifier.

9. The method of claim 2, further comprising:
causing the graphical user interface of the administrator user device to display an indication that the configurable message has been provided to the additional user device corresponding to the at least one user.

10. The method of claim 2, further comprising:
causing the graphical user interface of the administrator user device to display an indication that the at least one user has not viewed or interacted with the configurable message.

11. The method of claim 10, further comprising:
causing the graphical user interface of the additional user device to display a second configurable message associated with the potential health risk based at least in part on the indication that the at least one user has not viewed or interacted with the configurable message.

12. The method of claim 11, further comprising:
receiving, via the administrator user device, a user input to trigger the second configurable message, wherein causing the graphical user interface of the additional user device to display the second configurable message is based at least in part on receiving the user input.

13. The method of claim 2, further comprising:
identifying a second user of the one or more users who has not worn a respective wearable device associated with the second user for a time interval that satisfies a threshold time interval; and
causing the graphical user interface of the administrator user device to display a non-anonymized user identifier associated with the second user based at least in part on identifying that the second user has not worn the respective wearable device for the time interval.

14. The method of claim 13, wherein identifying the second user of the one or more users who has not worn the respective wearable device is based at least in part on a portion of the physiological data received from the respective wearable device associated with the second user, an absence of physiological data received from the respective wearable device associated with the second user, or both.

15. The method of claim 1, further comprising:
receiving one or more opt-in commands from one or more user devices associated with respective one or more users, wherein receiving the physiological data associated with the one or more users is based at least in part on receiving the one or more opt-in commands.

16. The method of claim 2, further comprising:
identifying one or more contributing factors for the potential health risk for the at least one anonymized user identifier; and
causing the graphical user interface of the administrator user device, the additional graphical user interface of the additional user device, or both, to display an indication of the one or more contributing factors.

17. The method of claim 2, wherein causing the administrator user device to display the notification of the potential health risk associated with the at least one anonymized user identifier comprises:
transmitting the notification to the administrator user device, wherein the notification comprises an email, a text message, a push notification, or any combination thereof.

18. An apparatus for health monitoring, comprising:
a processor;
memory coupled with the processor; and
instructions stored in the memory and executable by the processor to cause the apparatus to:
receive physiological data associated with one or more users, the physiological data being continuously collected via one or more wearable devices associated with each respective user of the one or more users, wherein the one or more users are associated with respective anonymized user identifiers;
identify one or more health risk metrics associated with each respective user of the one or more users based at least in part on a respective subset of the physiological data associated with each respective user; and
identify a potential health risk for at least one anonymized user identifier corresponding to at least one user based at least in part on the one or more health risk metrics associated with the at least one user satisfying one or more predefined thresholds.

19. The apparatus of claim 18, wherein the instructions are further executable by the processor to cause the apparatus to:
cause a graphical user interface of an administrator user device to display a notification of the potential health risk associated with the at least one anonymized user identifier; and
cause an additional graphical user interface of an additional user device corresponding to the at least one user to display a configurable message associated with the potential health risk.

20. The apparatus of claim 19, wherein the instructions are further executable by the processor to cause the apparatus to:
receive, via the administrator user device, a user input comprising the configurable message, wherein causing the additional user device to display the configurable message is based at least in part on receiving the user input.
